(19) United States
(12) Patent Application Publication
(10) Pub. No.: US 2010/0240979 A1
(43) Pub. Date: Sep. 23, 2010

(54) Device and System for Monitoring Blood Glucose Levels

(76) Inventor: Benjamin Atkin, North Miami Beach, FL (US)

Correspondence Address: ROBERT M. SCHWARTZ, P.A., P.O. BOX 221470, HOLLYWOOD, FL, 33022, US

(21) Appl. No.: 12/726,811

(22) Filed: Mar. 18, 2010

Related U.S. Application Data
(60) Provisional application No. 61/161,203, filed on Mar. 18, 2009.

(51) Int. Cl. A61B 5/145; G08C 19/00

(52) U.S. Cl. 600/365; 340/870.3

(57) ABSTRACT

A device and method for transmitting blood glucose level information from user provided glucometers, said glucometers originating from different manufacturers, to a receiver constructed and arranged to monitor, and sort said information and to selectively output said information.

### INDEX TO RELATED APPLICATIONS

This application claims benefit to U.S. Provisional Patent Application No. 61/161,203 filed Mar. 18, 2009, the disclosure of which is incorporated herein by reference in its entirety.

### BRIEF SUMMARY OF THE INVENTION

There is a need for a glucose management system that will be compatible with glucometers from different manufacturers. The present invention provides a device for receiving blood glucose measurement information from glucometers of different manufacturers, as well as compression, encryption, and SMS transmission of the blood glucose measurements to a blood glucose management system.

In one embodiment, the present invention is a device for receiving and transmitting blood

glucose measurement information comprising:
a. a mechanism for receiving blood glucose measurement information from a glucometer; and
b. transmitting mechanism;
wherein said transmitting mechanism sends information about the time, date, and said blood glucose level measured by said glucometer.

The device mechanism for receiving blood glucose measurement information utilizes electronics constructed and arranged to query a connected glucometer, wherein said query identifies said connected glucometer and provides a mechanism by which said glucometer transmits blood glucose measurement information from said glucometer to said device.

The device has a transmitting mechanism whereby information received from said glucometer is transmitted from said device. The transmission is by Internet transmission, wireless Internet transmission, Bluetooth, radio frequency, short message service (SMS), cell phone text message, direct transmission through a transmission cable, or combinations thereof.

In a preferred embodiment, the transmitting mechanism from said device is by short message service (SMS).

The device has a microprocessor; volatile memory (RAM); nonvolatile memory; meter interface electronics; a minimal user interface; an embedded GSM modem, which can be a cellular modem; an internal antenna; and a battery.

The blood glucose measurement, received by the device from a user-supplied glucometer, is processed as compressed data, encrypted using a proprietary algorithm, and transmitted by SMS.

The device will query a glucometer and the query includes a request for glucometer identification by manufacturer, confirmation of glucometer identification, and actuation of communication between said device and said glucometer.

The present invention is also a system for monitoring blood glucose measurements utilizing the device of the present invention and comprising:

a. a device constructed and arranged to receive blood glucose measurement information from a glucometer, and subsequently transmit information relating to day, time, and blood glucose measurements;
b. a receiver constructed and arranged to receive said transmitted information;
c. a computer readable medium associated with said receiver for compiling said transmitted information;
d. an electronic processor for sorting and arranged said transmitted information;
e. and output means for providing said transmitted information to a receiving entity.

The system utilizes the device that identifies a glucometer by manufacture and is constructed and said device arranged to communicate with glucometers from multiple manufacturers, said device, after said identification, subsequently queries, and communicates with said glucometer and receives blood glucose measurements.

### BRIEF DESCRIPTION OF THE DRAWINGS

FIG. 1 is a flow chart of the system and method of the present invention.

FIG. 2 is a glucometer positioned above a synchronizing cradle.

FIG. 3 is a glucometer positioned connected in the receiving cavity of a synchronizing cradle.

FIG. 4 is a receiving/transmission device configured with a male connector on one end to be paired with a female output connector on a glucometer and a USB connector on the end opposite the male connector.

FIG. 5 is a receiving/transmission device connected with a male connector to a female output connector on a glucometer and a USB connector on the end opposite the male connector.

### DETAILED DESCRIPTION OF THE PREFERRED EMBODIMENTS

The figures presented are for demonstrative purposes only. The device according to the present invention is any device constructed and arranged to function as described herein. The device of the present invention is constructed and arranged to query, identify, and communicate with a wide variety of glucometers regardless of the manufacture. The device receives information about blood glucose measurements from the glucometer, including information relating to the previous transmission of blood glucose measurements, the information is compressed, encrypted, and transmitted to a back end user utilizing the system and method of the present invention.

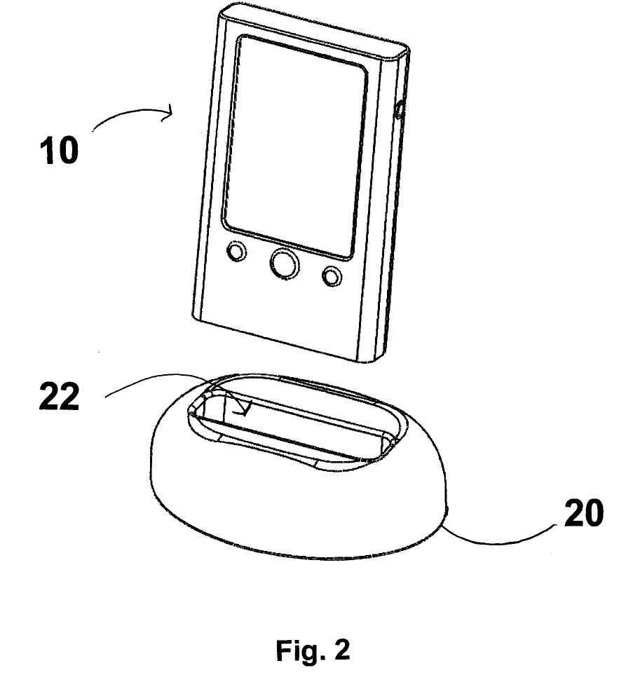

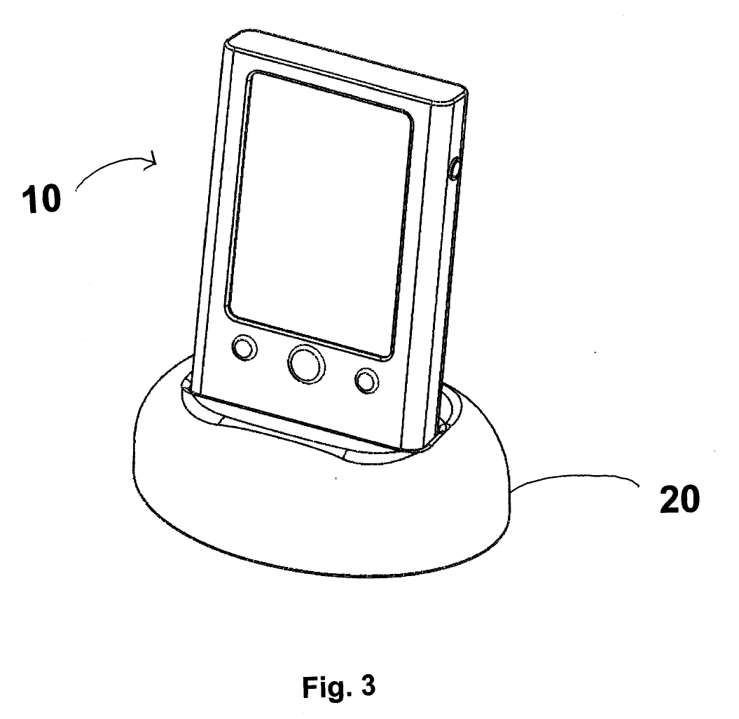

In one embodiment, shown in FIGS. 2 and 3, the device of the invention is a cradle 20. Cradle 20 has formed therein cradle cavity 22. Cradle cavity 22 is constructed and arranged to receive glucometer 10 therein. Glucometer 10 is a user supplied glucometer and may be from any manufacturer. Placing glucometer 10 in receiving cavity 22 of cradle 20 actuates cradle 20 to query and communicate with glucometer 10. Communication between cradles and cradled devices nested thereon is known in the art. The device and system of the present invention contemplates communication between cradle and cradled devices nested in conventionally known manners.

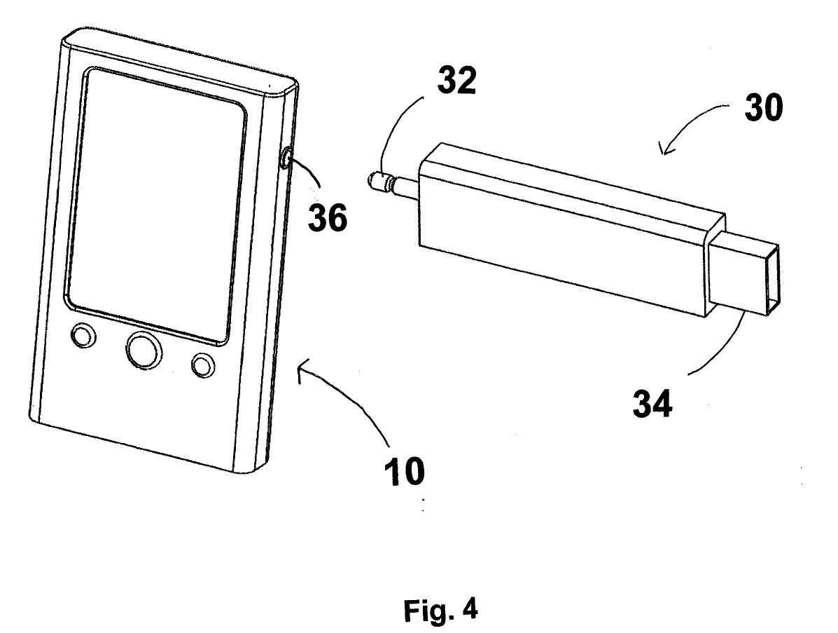

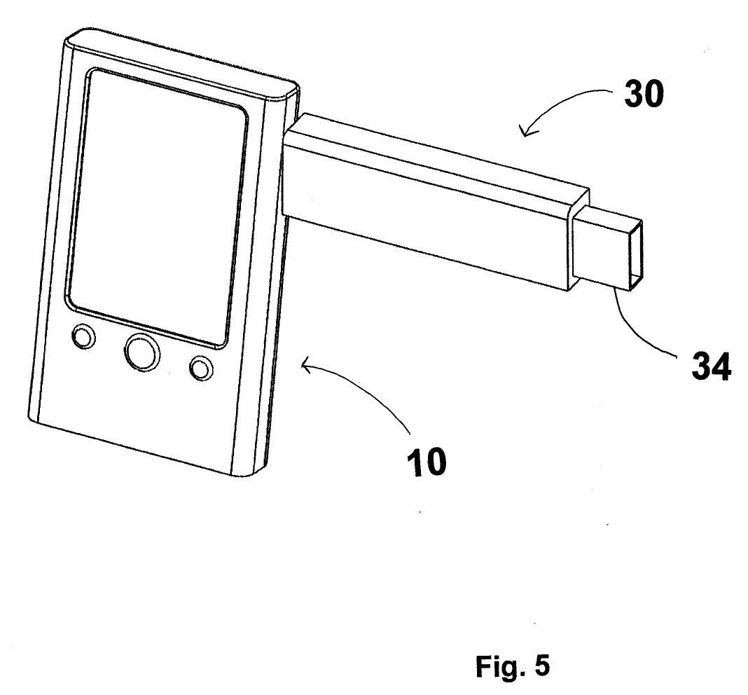

In one embodiment, shown in FIGS. 4 and 5, the device of the invention is a hand held plug-in device 30 constructed with a male communication port 32 compatible with a female communications port 36 on glucometer 10. Hand held plug in device 30 is a device of the invention as described herein and can communicate wirelessly after receiving blood glucose measurement information, compressing the information, and encrypting the received blood glucose measurement information. Optionally, either or both of cradle 20 or hand held plug-in device 30 has a USB or similar communications interface for connecting to a computer.

The device of the present invention is a stand alone, self-contained unit that will automatically query a user-supplied glucometer for blood glucose data (blood glucose and time/date values for stored blood glucose tests), receive said data, and transmit said data. The device is constructed and arranged to communicate with a wide variety of glucometers by utilizing electronics and stored information that will query and recognize a particular glucometer that is connected to the device.

The sending of data is by any appropriate means. The sending of data can be via one or more encrypted SMS text messages, either immediately or at some time in the future, to a central database server.

Additionally, the server can send one or more SMS text messages to the device for purposes of configuration or as part of the device's protocol for sending data to the server.

The device can employ a GSM data channel for purposes of remote firmware upgrade.

The system and method of the present invention will have server-side architecture and data handling appropriate to achieve the desired results of the present invention.

The device has a microprocessor, volatile memory (RAM), nonvolatile memory, meter interface electronics, a minimal user interface, an embedded GSM modem, which can be a cellular modem, an internal antenna, and a battery (primary or secondary). Where a secondary (rechargeable) battery is employed, the product also contains battery charging circuitry and a charging port. The device also includes one or more jacks, internal or external, to which one or more meter data cables will be connected.

### Microprocessor

The microprocessor will execute a custom program that effects the behaviors described in above and provides additional features such as power management.

The program will reside either in permanent or reprogrammable nonvolatile memory contained inside the microprocessor or in nonvolatile memory external to the microprocessor.

### Memory (RAM)

Depending on microprocessor selection, the device contains external volatile memory (RAM), or can make use of RAM contained inside the microprocessor.

### Nonvolatile Memory

Depending on microprocessor selection, the device also selectively contains external nonvolatile memory (e.g., flash), or can make use of nonvolatile memory contained inside the microprocessor.

The nonvolatile memory will contain various parameters essential to the operation of the device but subject to change device-to-device (e.g., a unique identifier); it can also contain the custom program itself (as with (7)).

### Meter Interface

The Meter Interface in the device of the present invention acts as the electrical intermediary between any given glucometer and the microprocessor (which must send data to and receive data from the glucometer). The electrical characteristics of each glucometer to be supported may differ (e.g., one can use a serial protocol with RS-232 signal levels, whereas another may use a serial protocol with TTL signal levels), and translation electronics will be needed to enable the microprocessor to communicate with different glucometers.

Not all glucometer data ports have the same physical interface (e.g., one may use a 2.5 mm male “stereo” TRS (tip-ring-sleeve) plug, whereas another may use a 2.5 mm male TRRS (tip-ring-ring-sleeve) plug); the Meter Interface will permit one of several different types of physical glucometer interfaces to be presented to the user's glucometer.

### GSM Modem

The device will contain an embedded GSM (Global System for Mobile communication) modem, enabling communication to the server through a GSM cellular network via SMS text messages and potentially a network data stream.

The device will contain a SIM card programmed to permit access to the GSM network by the device; the SIM card will identify the device to the network for purposes of accounting and billing for network usage.

### Battery

The device's sole power source will selectively be a primary (nonrechargeable), a secondary (rechargeable) battery, or combinations thereof.

### User Interface

The device will have a simple user interface to communicate certain important information to the user, potentially including:
a. Device activity;
b. Communication success or failure;
c. GSM network and connectivity status;
d. Low battery condition.
e. Completion of charging (if secondary batteries are used)

### Detection of Connected Meter

The device can periodically attempt to discover a glucometer connected to the device by “polling” for known meters (that is, by attempting to send data that should cause a given meter to respond with its own data, inferring the absence of that meter after some time elapses without a response), or it can electronically detect the connection of a meter and only poll for some period of time after a connection appears to have been made.

### Download of Data

When a connected glucometer responds to a poll, the device will read and validate the full response.

If a response is valid, the device will extract a fixed or variable number of the most recent blood glucose test results (including blood glucose value and the time and date of the test).

If the device is configured to send results immediately, it will create and send to the server an SMS message containing a fixed or variable number of the most recent blood glucose test results.

If the device is configured to send results at a later time, it will store the extracted data and send the SMS message at the configured time or around the configured time (if a random algorithm is employed to reduce server-side SMS message processing load).

### Transmission of Data

When data is to be sent, blood glucose report results along with identifying and other information will be bit-wise packed or otherwise encoded into a temporary memory buffer.

The prepared buffer will then be encrypted using an appropriate encryption algorithm to ensure that the user's medical data cannot be casually, accidentally, or intentionally intercepted and interpreted.

A fixed length CRC (cyclic redundancy check) value will then be calculated for and appended onto the encrypted buffer.

The resulting buffer will then be transformed into an SMS message in such a way as to make maximum use of the available data bandwidth of the SMS messaging channel.

The SMS message will then be transmitted to the GSM modem.

### Transmission Reliability Protocol

The device can employ a two-way messaging protocol with the server (e.g., ACK/NAK with timeouts and retries) to ensure that the message was received by the server with no errors.

### Communication From Server

The device can expect and respond to SMS messages sent from the server for the purposes of configuration, device identification, device activation, firmware upgrade (see below), or other purposes.

### Firmware Upgrade

The device can support the remote update of its internal firmware by opening a GSM network data channel to the server and requesting the download of new firmware.

One example of a preferred embodiment is set forth as follows: A user will pair a glucometer with a transmission device. The transmission device will connect with the glucometer in either direct of wireless connection. The transmission device will upload blood glucose measurements from the device, compress the data, encrypt the data, and send the data as an SMS message.

In one embodiment, the transmission device has no user operated buttons, actuators, or the like. The transmission device is similar to “plug and play” computer devices in that once connection is established, the transmission device is constructed and arranged to function as desired. An important aspect of the transmission device is the configuration whereby the device can query, recognize, and receive data from a wide variety of glucometers regardless of the manufacturer of any particular glucometer.

### Report Data

Each individual SMS blood glucose report will contain the following information (along with other protocol-related data discussed later):

Unique Subscriber ID (not related to GSM subscriber ID)

Most recent 20 (or fewer) BG readings, comprising:
Timestamp (DD/MM/YY HH:mm)
Blood Glucose Value

Individual report fields are detailed below:

| | | Bit |
|---|---|---|
| Field | Range | Count |
| Subscriber | [0, | 28 |
| ID | 268435455] | |
| Year | [0, 15] (0 = | 4 |
| | 2009) | |
| Month | [1, 12] | 4 |
| Day | [1, 31] | 5 |
| Hour | [0, 23] | 5 |
| Minute | [0, 59] | 6 |
| Blood | [1, 512] | 9 |
| Glucose | | |

### Encoding

The maximum SMS message length (employing 8-bit ASCII) is 140 characters. (160 characters are available with 7-bit ASCII.) 95 printable ASCII characters (including space) are available if internationalized glyphs are not employed (111 if internationalized glyphs are employed). Since these numbers are short of a full 7-bit range (128), the encoding scheme will use 64 symbols, yielding a payload of 6 data bits per SMS symbol, for a total of 840 available bits (exactly 105 bytes' worth). (Note that if using 7-bit ASCII/160 characters, a total of 960 bits, or 120 bytes, are available.)

The symbols A-Z, a-z, 0-9, @, and $ will be used, in that order, as the transmittable SMS representation for binary sextet values [0, 63].

To ensure the validity of transmitted data, the last 16 available bits will be used to store a CRC-16 value computed for the encoded buffer (except for the CRC-16 bits themselves).

To provide protocol version management, the first 8 bits will be used to convey a protocol version number, to give the back-end the ability to adapt to any necessary changes to the protocol that occur over time.

The encoding algorithm comprises three 1 steps:
1. Encode protocol version and report data into an intermediate encoding buffer.
   The bit values for each field will first be concatenated (contiguously) into the first 824 bits (103 bytes) of the intermediate buffer, sized to hold 840 bits (105 bytes).
2. Calculate CRC-16 and insert at end of intermediate encoding buffer.
   The CRC-16 will then be calculated and placed in the last 16 bits (2 bytes) of the intermediate buffer.
3. Translate bit sextets from intermediate encoding buffer into SMS symbols in an SMS buffer.
   Each bit sextet from the intermediate buffer will be translated into a transmittable SMS symbol and placed into an SMS buffer.

Storage of values in the intermediate and SMS buffers will be little-endian.

The entire encoding algorithm is illustrated below:
Error! No topic specified.

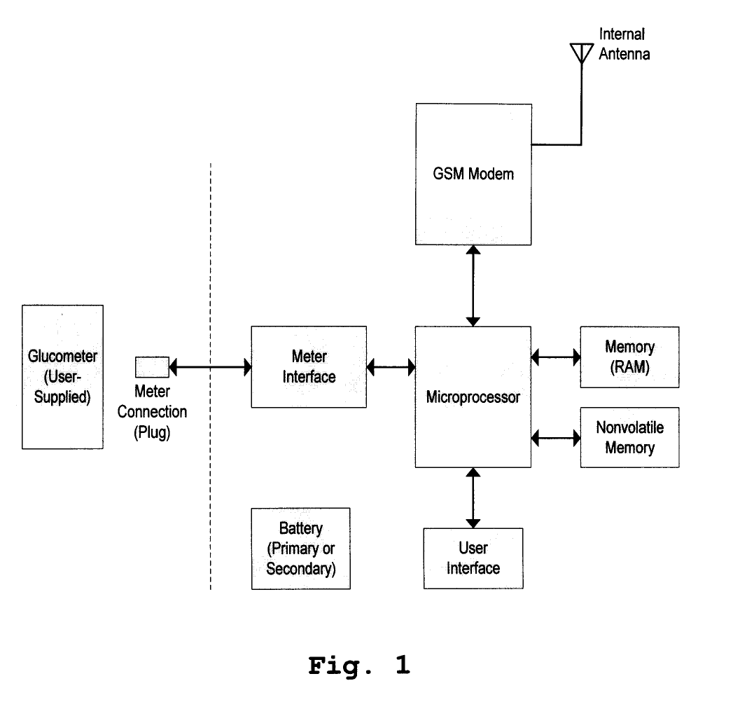

FIG. 1—Intermediate Encoding and CRC-16
Error! No topic specified.

FIG. 2—Final SMS Encoding

In the translation step, sextets are transformed into SMS symbols as follows:

| Sextet | SMS |
|---|---|
| Code | Symbol |
| 0-25 | A to Z |
| 26-51 | a to z |
| 52-61 | 0 to 9 |
| 62 | @ |
| 63 | $ |

After translation, the resulting SMS buffer will be transmitted, LSB-first (little-endian), as a 140 character SMS message. (E.g., in the example SMS buffer in the figure above, the first 6 characters of the SMS message will be “rJX@9m”.)

### CRC-16 Algorithm

One example of a CRC-16 algorithm is included below as C code for reference. If needed, a table-driven implementation could also be employed.

uint8 interimBuf[105]; // <-- the intermediate encoding
buffer
uint8 ndx = 0;
uint16 crc = 0;
do
{

uint8 i;
crc \hat{ }= (uint16)interimBuf[ndx++] << 8;
for (i = 0; i < 8; ++i)
{

if (crc & 0x8000)

crc = (crc << 1) \hat{ } 0x1021;

else

crc <<= 1;

}

} while (ndx < 103);

As stated above, the system of the present invention has a device constructed and arranged to query, recognize, and receive data from a wide variety of glucometers regardless of the manufacturer of any particular glucometer.

Meters from different manufacturers are recognized in two ways: first, by physical electronic interface (some meters employ RS-232 signaling voltage levels in their serial interface; others employ standard logic levels), and second, by the specifics of the data protocol (meters from a specific manufacturer—or even specific models of meter from the same manufacturer—will respond correctly to a specific protocol; other manufacturers' meters will either not respond or will respond with an error condition).

The system of the present invention includes a device capable of recognizing different glucometer models and manufacturers. This recognition aspect is unique because there are differences in how data is retrieved from different meters from different manufacturers.

The system of the present invention required research and development though engineering and experimentation to identify the parameters of the physical interface specific to different glucose meters (glucometers). For example, mechanical differences: what physical plug type, how many conductors, what signals on what conductors; electrical: logic or RS-232 levels, any other signal lines?). After the research and engineering, a device was constructed to implement the meter specific data in a communication protocol.

In order to interact with multiple meters, some using logic level serial signaling and some RS-232 level, the present invention had to have a device with integrated software having a ability to allow the software to select either logic level or RS-232 level signaling, depending on which meter it was either attempting to probe or was actively communicating with. The device is required to have such a software interface or it would not be able to communicate with a wide variety of glucometers.

To clarify, assume that all meters' protocols have some sort of “Identify yourself” message, to which the meter will respond with some kind of response data; let's say that meter ‘A’ employs logic level signaling and meter ‘B’ employs RS-232 level signaling; further, meter ‘A’ mates with plug type ‘A’, and meter ‘B’ mates with plug type ‘B’, where plugs ‘A’ and ‘B’ are not interchangeable (slightly different barrel sizes, perhaps).

(Note: this one manner of ensuring that RS-232 voltage levels are not accidentally applied to a meter that expects logic level signals, which could potentially damage the meter.)

In device used in the system of the present invention, the logic level serial signals (input and output) are made available to adapter plugs in two forms: first, the raw serial logic signals coming from the microprocessor; and second, RS-232 level signals coming from an RS-232 level shifter between the adapter plug and the microprocessor. Plug type ‘A’ connects the logic level signals to its meter-side plug, and plug type ‘B’ connects the RS-232 level signals to its meter-side plug.

In this scheme, the firmware in the microprocessor could disable the RS-232 level shifter while it is communicating with or attempting to communicate with (probe) a meter that does not use RS-232 levels.

In one embodiment, the device of the present invention communicates through direct connection into a glucometer data port. The direct connection is a plug configuration or a cradle connection base configured for connection. In the embodiment where a cradle type connection is used, the cradle is the device for receiving information from the glucometer and transmitting the received data according to the present invention.

Once the device communication is established between the device of the present invention and the user supplied glucometer, the device initiates a data compression step. Data compression is applied first, necessarily so. Most data encryption schemes will increase the entropy (Shannon entropy, or information entropy) of a given set of data, which limits compressibility; therefore compression, which also increases entropy but reduces the size of the data. The encryption is contemplated to be compliant with HIPPA and other standards relating to privacy requirements of patient medical information.

In one embodiment of the present invention, an asymmetric key cryptography (e.g., RSA) would be employed to ensure data security. Since the size of data being transmitted is rather small (140 characters maximum for SMS, ignoring some carriers' provision for permitting 160 characters), the encryption time is kept to a minimum.

The present invention successfully addressed another obstacle that has not been overcome. Blood glucose and other data must be packed into a buffer (of some maximum size, discussed below) in such a way as to make most efficient use of the available bits, since SMS places a limit on the amount of information that may be transmitted in a single message. Since there is a hard limit, it was not desirable to employ a lossless data compression algorithm (e.g., Lempel-Ziv or similar), because such compression algorithms could not necessarily guarantee that the compressed data would always fit inside the buffer size limit.

Instead, the present invention utilizes a compression to “pack” data into bytes, depending on how many bits were needed for each datum. E.g., for timestamps, the month value takes on 12 different values (‘1’ through ‘12’); this datum can be fully represented with four bits.

SMS text messages are limited to a maximum of 140 characters. However, these are SMS characters, not full bytes. Based on the actual number of printable ASCII characters (including space) available in an SMS text message (assuming internationalized glyphs are not employed), there are 95 different symbols (characters) that we can use. Since this is a little short of a full 7-bit range (128), we chose to use a subset of 64 symbols, yielding a payload of 6 data bits per SMS symbol, for a total of 840 available bits (exactly 105 bytes).

After the medical data (glucose measurements, day and time of measurement) is retrieved by the device has been packed into a 105-byte data buffer (data compression or data packing phase) and encrypted, there is a data encoding phase that takes each group of 6 bits and “encodes” them into a single SMS symbol (character). This process results in a single SMS text message of exactly 140 characters in length.

Once the final SMS message has been produced, the message is conveyed to a recipient and the SMS recipient to an embedded GSM GPRS module, which then transmits an SMS message to the GSM network.

When the SMS message is received by the back-end server, the steps discussed previously must be un-done in reverse order to yield subscriber information, time stamped blood glucose results, and so forth (decoding, decryption, unpacking, in that order).

Once the back-end has the final, raw data values, however, standard and conventional processing occurs (which includes aspects including, but not limited to subscriber verification, recording of new readings and other data in a database, and the like).

The present invention further contemplates a single device employed with multiple glucometers. For example, in a hospital or nursing home setting, where 20 patients in a certain area have glucometers, a single device will individually transmit the glucometer data according to the system and method of the present invention, and the back end receiver will differentiate each patient based on the glucometer identifier transmitted with the glucometer readings.

In one embodiment, the back end server is a system for receiving processing data transmitted by the device of the present invention. The back end server is part of the system and method of the present invention and may be configured as desired to further transmit information to a physician, health care provider, monitoring service, insurance company, email, cell phone, or combinations thereof. Data may be sent in tabular, statistical, graphical presentations, or combinations thereof, as well as selectively transmitting individual glucose measurements.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A device for receiving and transmitting blood glucose measurement information comprising:
a. a mechanism for receiving blood glucose measurement information from a glucometer; and
b. transmitting mechanism;
wherein said transmitting mechanism sends information about the time, date, and said blood glucose level measured by said glucometer.

2. The device of claim 1 wherein said mechanism for receiving blood glucose measurement information utilizes electronics constructed and arranged to query a connected glucometer, wherein said query identifies said connected glucometer and provides a mechanism by which said glucometer transmits blood glucose measurement information from said glucometer to said device.

3. The device of claim 1 wherein said transmitting mechanism from said device is Internet transmission, wireless Internet transmission, Bluetooth, radio frequency, short message service (SMS), cell phone text message, or direct transmission through a transmission cable.

4. The device of claim 1 wherein said transmitting mechanism from said device is by short message service (SMS).

5. The device of claim 1 wherein the device comprises a microprocessor; volatile memory (RAM); nonvolatile memory; meter interface electronics; a minimal user interface; an embedded GSM modem, which can be a cellular modem; an internal antenna; and a battery.

6. The device of claim 1, wherein said blood glucose measurement is processed as compressed data, encrypted using a proprietary algorithm, and transmitted by SMS.

7. The device of claim 1, wherein said query includes a request for glucometer identification by manufacturer, confirmation of glucometer identification, and actuation of communication between said device and said glucometer.

8. A system for monitoring blood glucose measurements comprising:
a. a device constructed and arranged to receive blood glucose measurement information from a glucometer, and subsequently transmit information relating to day, time, and blood glucose measurements;
b. a receiver constructed and arranged to receive said transmitted information;
c. a computer readable medium associated with said receiver for compiling said transmitted information;
d. an electronic processor for sorting and arranged said transmitted information;
e. and output means for providing said transmitted information to a receiving entity.

9. The system of claim 8, wherein said device identifies a glucometer by manufacture and is constructed and said device arranged to communicate with glucometers from multiple manufacturers, said device, after said identification, subsequently queries, and communicates with said glucometer and receives blood glucose measurements.
